(19) United States
(12) Patent Application Publication
(10) Pub. No.: US 2004/0110274 A1
(43) Pub. Date: Jun. 10, 2004

(54) Apparatus for stacking, repositioning, agitating and knocking

(76) Inventor: Ilya Feygin, Mountainside, NJ (US)

Correspondence Address: DEMONT & BREYER, LLC, SUITE 250, 100 COMMONS WAY, HOLMDEL, NJ, 07733, US

(21) Appl. No.: 10/725,988

(22) Filed: Dec. 2, 2003

Related U.S. Application Data
(60) Provisional application No. 60/430,255, filed on Dec. 2, 2002.

(51) Int. Cl. C12M 1/00

(52) U.S. Cl. 435/283.1; 435/289.1

(57) ABSTRACT

An apparatus that is capable of performing at least some of the physical operations that are used in conjunction with growing cells in cell-cultivation flasks. The operations performed include one or more of the following: receiving a plurality of cell-cultivation flasks, re-orienting the flasks; agitating liquid within the flasks, and knocking the cell-cultivation flasks to loosen cellular material within the flasks. In accordance with the illustrative embodiment, re-positioning is implemented using a movable platform that cooperates with guides, and a drive that moves the movable platform. In some embodiments, the guides are implemented as slots in a frame. In some embodiments, a single drive is used to conduct all operations.

### STATEMENT OF RELATED CASES

This case claims priority of U.S. Provisional Patent Application Serial No. 60/430,255, filed Dec. 2, 2002.

### FIELD OF THE INVENTION

The present invention relates to an apparatus for performing operations that are required or otherwise desirable in conjunction with growing cells within a cell-cultivating flask.

### BACKGROUND OF THE INVENTION

Developments in cellular biology and related fields have led to increased demands for devices capable of producing cells. The cells are often used, for example, to produce biologically-active compounds.

For growing static cell cultures, flat, flask-like containers (“cell-cultivating flasks”) are typically used. The flasks usually incorporate a neck and an opening that is closed using a screw cap. Some flasks in the prior art incorporate internal structures for cultivating the cells, while others do not.

After adding a cultivating medium and cells to the flask, it is sometimes necessary to conduct certain physical operations. These operations include, for example, changing the orientation of the flask (e.g., from a horizontal position to a vertical position, or visa-versa, etc.) and agitating the flask. Furthermore, on the completion of cell growth, and prior to emptying the cell-cultivating flasks, it is sometimes necessary to “knock” or shake the flask to loosen cellular material that otherwise adheres to the internal walls of the flask.

Traditionally, these physical operations have been performed manually. Such manual operations limit production and increase production expenses. As a consequence, the art would benefit from an apparatus that can automatically perform these operations.

### SUMMARY

The illustrative embodiment of the present invention is an apparatus that is capable of performing at least some of the physical operations that are used in conjunction with growing cells in cell-cultivation flasks. The physical operations performed by an apparatus in accordance with the illustrative embodiment include one or more of the following functions, in addition to any others:

Receiving, in a stack, a plurality of cell-cultivation flasks, wherein the flasks can be fed manually or by a robotic system to the apparatus.

Re-orienting the flasks from a horizontal position to a vertical position or visa versa.

Agitating liquid within the flasks, advantageously creating a circular flow of liquid.

“Knocking” the cell-cultivation flasks to loosen cellular material within the flasks.

In accordance with the illustrative embodiment, horizontal-to-vertical re-positioning is implemented using a movable platform that cooperates with guides. In use, cell-cultivating flasks are stacked on the movable platform.

In the illustrative embodiment, the guides are implemented as slots in a frame. More particularly, in some embodiments, the frame includes two, spaced plates, each of which has a generally horizontal base and a generally vertical riser, wherein the riser depends from one end of the base. A slot is defined in both the base portion and the riser portion of each plate.

The slot in the base is horizontal, while the slot in the riser takes the form of a vertical arc. In the illustrative embodiment, two sets of rollers depend from the platform; one set (proximal to a first end of the platform) engages the slot in the base and the other set (distal to the first end of the platform) engages the slot in the riser. Moving the platform towards the riser causes the set of rollers that cooperate with the slot in the riser to ride upwards in a vertical arc. This causes the second end of the platform to move upward. Since the first end of the platform does not move in the vertical direction due to its engagement with the horizontal slot, the platform rotates about the first end toward a vertical orientation. The motion of the platform is actually a combination of sliding and rotating, since the platform moves horizontally toward the riser while the second end rises. With continued movement toward the riser, the platform attains full vertical orientation, having rotated ninety degrees from its horizontal orientation. Cell-cultivating flasks that are stacked on the platform are, of course, rotated along with the platform.

The use of the slotted frame and cooperating movable platform, in accordance with the illustrative embodiment, enables the apparatus to maintain the same bottom reference plane. That is, at least part of the movable platform is always engaged to horizontal slot in the base, which defines a bottom reference plane. This is in contrast to a classic mechanism for rotating an object, which utilizes a “twisting” action. The classic mechanism typically does not maintain the bottom reference plane, and, in fact, has to be lifted to enable an object to be rotated (assuming that the object is resting upon a surface).

In accordance with the illustrative embodiment, the slot in the riser of one of plates and a slot in the base of the other of the two plates include a deviated portion. The deviated portion causes a local change in the height of slots. Moving the movable platform back-and-forth such that it successively passes the deviated portions in the two slots causes a wobbling motion in the platform—and in any cell-cultivating flasks that are disposed on the platform. This wobbling motion places liquid within the flasks in orbital (i.e., circular) motion.

The slots in the frame are, therefore, dual functional, in the sense that they enable both the re-positioning and agitation functions. And as a consequence of this dual functionality, a single actuator can be used to drive both the re-positioning and agitating operations.

In some embodiments, the apparatus also incorporates a spring-loaded “hammer” mechanism that generates an adjustable and repeatable force (a “knocking”) that is directed against the side of the cell-cultivating flasks. In some embodiments, the spring-loaded hammer is implemented as a passive device, wherein the energy that loads the spring is imparted by the actuator that drives the re-positioning (and agitating) operation. An embodiment of the spring-loaded “hammer” mechanism is described in applicant's co-pending patent application entitled “Passive Force-Imparting Mechanism” (Attorney Docket No. 153-035, filed 02 Dec. 2003), incorporated by reference herein.

### BRIEF DESCRIPTION OF THE DRAWINGS

FIG. 1 depicts an apparatus 100 in accordance with the illustrative embodiment of the present invention, wherein a plurality of cell-cultivating flasks, which are stacked on the apparatus, are in a horizontal orientation.

FIG. 2 depicts the apparatus of FIG. 1, wherein the cell-cultivating flasks are in a vertical orientation.

FIG. 3 depicts the apparatus of FIG. 1, but without cell-cultivating flasks present. Furthermore, FIG. 3 depicts drive mechanism 330 of apparatus 100.

FIG. 4 depicts the apparatus of FIG. 3.

FIG. 5 depicts a flow of liquid within a cell-cultivating flask during agitation.

### DETAILED DESCRIPTION

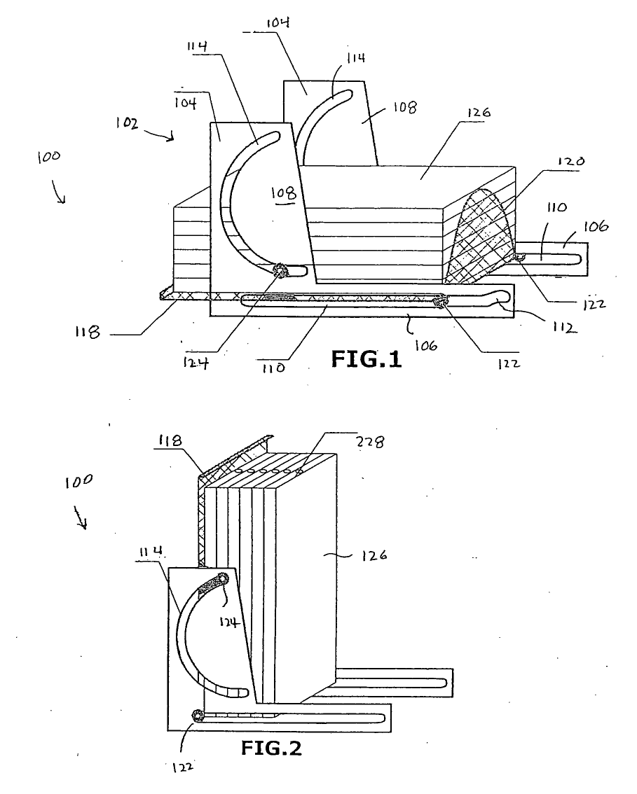

FIG. 1 depicts apparatus 100, which includes frame 102 and platform 118, which cooperate as depicted.

In the illustrative embodiment, frame 102 comprises two, identical plates 104. Each plate 104 has a generally horizontal base 106 and a generally vertical riser 108. The riser depends from base 106 near one end thereof. In some embodiments, bases 106 are attached to an underlying platform, which is not depicted. The underlying platform can be used, for example, to support a spring-loaded hammer mechanism to implement a knocking operation.

Each base 106 includes slot 110, which has a substantially horizontal orientation. Each riser 108 includes arcuate-shaped slot 114, which has a substantially vertical orientation. As used herein, the term “substantially horizontal orientation” means a near-horizontal orientation of the slot, such as 0 degrees +/−about 5 degrees. As used herein, the term “substantially vertical orientation” means that, relative to the horizontal (e.g., relative to slot 110), slot 114 is angled upwards at an angle of at least about 45 degrees (wherein the angle is defined by two intersecting rays, one of which is coincident with slot 110 and the other of which passes through the end points of slot 114).

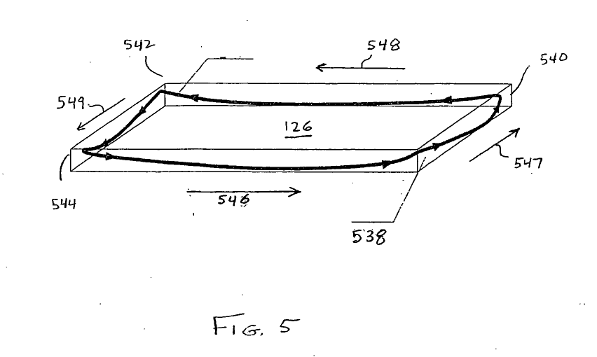

Slot 110 in base 106 of one of plates 104 includes deviated portion 112. Vertically-oriented slot 114 in riser 108 of the other of plates 104 includes deviated portion 116. (Only deviated portion 112 is visible in FIG. 1; see FIGS. 4 and 5 for deviated portion 116.)

In the illustrative embodiment, deviated portion 112 is displaced upward relative to the rest of slot 110 and deviated portion 116 is displaced upward relative to the immediately surrounding portion of slot 114. In some other embodiments (not depicted), each deviated portion 112,116 is displaced downward. As described more fully later in this specification, deviated portions 112 and 116 are used during agitation operations to impart circular motion to a liquid being agitated within cell-cultivating flasks 126.

Platform 118 receives cell-cultivating flasks 126. In the illustrative embodiment, platform 118 includes retainer 120, which depends from the front end of the platform (i.e., the “right” end as viewed in FIG. 1). As described further below, retainer 120 is used in conjunction with re-positioning operations.

Platform 118 is movable relative to frame 102 to facilitate re-positioning and agitating operations. In the illustrative embodiment, platform 118 is physically adapted to slide within frame 102. This capability is provided, in the illustrative embodiment, by two sets of rollers that cooperate with slots 110 and 114. More particularly, platform 118 includes a set of front rollers 122 and a set of rear rollers 124. Front rollers 122 engage and are guided by slots 110 in bases 106. Similarly, rear rollers 124 engage and are guided by slots 114 in risers 108.

The cooperating arrangement of rollers 122, 124 and slots 110, 114 are sufficient to keep plates 104 of frame 102 in spaced, parallel relation to one another. In some variations of the illustrative embodiment (not depicted), individual sections 104 are linked to one another at risers 108 by one or more beams to provide additional rigidity.

As indicated above, in use, one or more flasks 126 are loaded onto platform 118. Flasks 126, which in the illustrative embodiment are flat flasks, each include a port, which will typically having a protruding “neck” or “throat” for receiving liquid. (Not depicted in FIG. 1, see, FIG. 2: “ports 228”.) FIG. 1 depicts six flasks 126 on platform 118, although a greater number or less number of flasks can be accommodated. The flask loading operation can be conducted either manually or automatically, in conjunction with materials-handling equipment (not depicted).

1. Re-Positioning Function

With reference to FIG. 2, re-orientation (vertical positioning) of flasks 126 is achieved by forcing platform 118 towards the rising curve of slots 114 until both rear rollers 124 and front rollers 122 reach their limits of travel. When flasks 126 are in a vertical position, ports 228 are oriented “upwards” to be accessed by a fluid-delivery mechanism. (The fluid-delivery mechanism is not a part of this invention and is not described or depicted herein.) Retainer 120 supports plates 126 when platform 118 is in a vertical position.

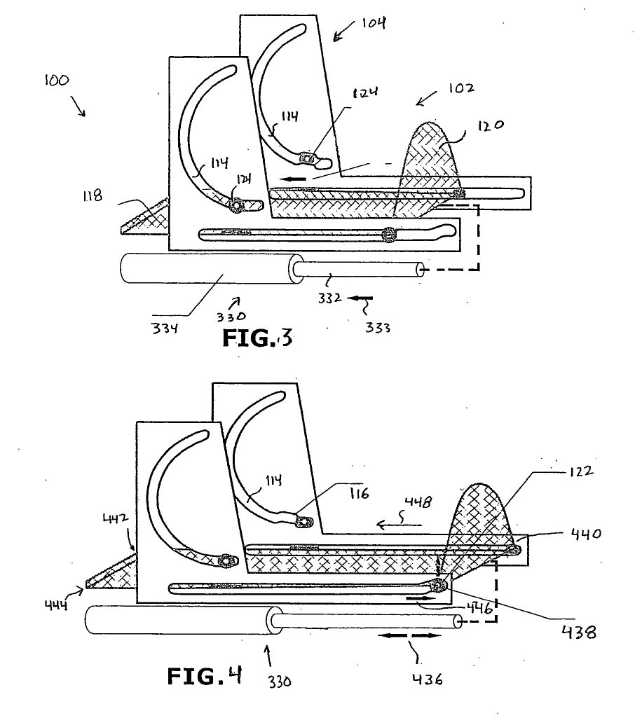

Platform 118 is moved via a drive mechanism, which can be implemented in many different ways. In the illustrative embodiment depicted in FIG. 3, drive mechanism 330 is implemented as a linear actuator. The linear actuator is realized, in the illustrative embodiment, as an air-actuated piston/cylinder.

Drive mechanism 330 is secured to a surface or platform that underlies bases 106 of frame 102. Piston 332 of drive mechanism 330 is coupled to front (right) end of platform 118 (i.e., near retainer 120). Drive mechanism 330 is appropriately positioned so that as piston 332 is retracted in direction 333 into cylinder 334, platform 118 moves toward generally vertically-oriented slots 114. As it does so, rollers 124 ride up slots 114 and rollers 122 travel toward the back of slots 110. This movement re-positions platform 118 and any flasks 126 that are disposed on it, into the vertical orientation depicted in FIG. 2.

Thus, in the illustrative embodiment, slots 110 and 114 in respective x and z planes convert the linear and horizontal movement of drive mechanism 330 into rotational movement of platform 118.

2. Agitation Function

Any liquid that is present in flasks 126 can be agitated by moving platform 118 back and forth in a reciprocating motion. Furthermore, by virtue of deviated portions 112 and 116, such liquid can advantageously be agitated into circular wave motion.

Referring now to FIGS. 4 and 5, moving platform 118 back and forth along vector 436 using drive mechanism 330 will cause liquid to move back and forth in wave motion within flasks 126. FIG. 5 depicts path 546 of liquid within a flask 126.

As platform 118 begins to move toward the “right” along direction 446 (FIG. 4), liquid flows along path 546 toward the “right” side of flask 126 (FIG. 5). As platform 118 moves further rightward, roller 122 rides up deviated portion 112 of slot 110. This causes “forward” right corner 438 of platform 118 to rise relative to “rearward” right corner 440. Consequently, platform 118 is tilted toward corner 440. As depicted in FIG. 5, this causes liquid within flask 126 to move from relatively-higher corner 538 to relatively-lower corner 540 along path 547.

As platform 118 begins to move back toward the left along direction 448 (FIG. 4), liquid flows toward the “left” side of flask 126 along path 548 (FIG. 5). As platform 118 moves further leftward, roller 124 rides up deviated portion 116 of slot 114. This causes “rearward” left corner 442 to rise relative to “forward” left corner 444. Consequently, platform 118 is tilted toward corner 444. As depicted in FIG. 5, this causes liquid within flask 126 to move from relatively-higher corner 542 to relatively-lower corner 544 along path 549.

In this fashion, circular wave motion is created in flasks 126. This agitation pattern can be advantageous as a function of the particular processes that are occurring within flasks 126.

It is to be understood that the above-described embodiments are merely illustrative of the present invention and that many variations of the above-described embodiments can be devised by those skilled in the art without departing from the scope of the invention. It is therefore intended that such variations be included within the scope of the following claims and their equivalents.

I claim:

1. An apparatus comprising:
a first slot having a substantially horizontal orientation;
a second slot having a substantially vertical orientation;
a platform, wherein said platform has a first end, and wherein said platform is movably coupled to said first slot proximal to said first end and movably coupled to said second slot distal to said first end; and
a drive, wherein said drive is coupled to and moves said platform.

2. The apparatus of claim 1 further comprising:
a first member and a second member that depend from said platform, wherein:
said first member depends from said movable platform at a location that is proximal to said first end of said platform;
said second member depends from said movable platform at a location that is distal to said first end of said platform;
said first member engages said first slot; and
said second member engages said second slot.

3. The apparatus of claim 1 wherein said drive is a linear drive.

4. The apparatus of claim 1 wherein said second slot has an arcuate shape.

5. The apparatus of claim 2 wherein said first member is a roller and said second member is a roller.

6. The apparatus of claim 1 further comprising a frame, wherein said frame comprises a first plate and a second plate that are spaced-apart from one another, wherein said first slot and said second slot are defined in said first plate, and a third slot that is identical to said first slot and a fourth slot that is identical to said second slot are defined in said second plate.

7. The apparatus of claim 6 wherein said first slot has a first deviated portion that is displaced in a vertical direction relative to the remainder of said first slot.

8. The apparatus of claim 7 wherein said fourth slot has a second deviated portion that is displaced vertically relative to a proximal portion of said fourth slot.

9. The apparatus of claim 6 wherein said first plate and said second plate each comprise a horizontally-extending base and a vertically-extending riser, and wherein said first slot is defined in said base and said second slot is defined in said riser of said first plate, and further wherein said third slot is defined in said base and said fourth slot is defined in said riser of said second plate.

10. An apparatus comprising:
a movable platform;
a linear drive, wherein said linear drive is coupled to and moves said movable platform in linear motion; and
a guide, wherein said movable platform is physically engaged to said guide, and further wherein said guide is physically configured to convert said linear motion of said linear drive to rotational motion that causes said movable platform to move between a horizontal orientation and a vertical orientation.

11. The apparatus of claim 10 wherein said guide is further physically configured to cause said movable platform to wobble as said linear drive moves said movable platform.

12. The apparatus of claim 10 wherein said movable platform is physically adapted to receive a plurality of flat flasks, wherein, when received, said flat flasks are arranged in a stack.

13. The apparatus of claim 10 wherein said guide comprises:
a first slot having a substantially horizontal orientation; and
a second slot having a substantially vertical orientation.

14. The apparatus of claim 13 wherein said second slot has an arcuate shape.

15. The apparatus of claim 13 wherein said guide comprises a frame, and wherein said frame comprises a first plate and a second plate that are spaced-apart from one another, and further wherein said first slot and said second slot are defined in said first plate.

16. An apparatus comprising:
a movable platform;
a drive, wherein said drive is coupled to and moves said movable platform in linear motion; and
a guide, wherein said movable platform is physically engaged to said guide, and further wherein said guide comprises physical adaptations for:
converting said linear motion imparted by said drive to rotational motion that causes said movable platform to move between a horizontal orientation and a vertical orientation; and
causing said movable platform to wobble as said drive moves said movable platform.

17. The apparatus of claim 16 said movable platform is physically adapted to receive a plurality of flat flasks, wherein, when received, said flat flasks are arranged in a stack.

18. The apparatus of claim 16 wherein said physical adaptation for converting aid linear motion to rotational motion comprises:
a first slot having a substantially horizontal orientation; and
a second slot having a substantially vertical orientation.

19. The apparatus of claim 18 wherein said second slot has an arcuate shape.

20. The apparatus of claim 18 wherein said physical adaptation for causing said movable platform to wobble comprises a first deviated portion in said first slot that is displaced in a vertical direction relative to the remainder of said first slot.

21. An apparatus comprising:
a frame, wherein said frame has two spaced-apart plates, and wherein each said plate comprises a substantially horizontally-oriented slot and a substantially vertically-oriented slot;
a platform, wherein said platform is movably coupled to said frame at said substantially horizontally-oriented slots and at said substantially vertically-oriented slots;
a drive mechanism, wherein:
said drive mechanism is coupled to said platform; and
said drive mechanism moves said platform.

22. The apparatus of claim 21 wherein one of said substantially horizontally-oriented slots and one of said substantially vertically-oriented slots include a physical adaptation that causes said platform to wobble as said drive moves said platform.
